(19) United States
(12) Patent Application Publication
(10) Pub. No.: US 2003/0199476 A1
(43) Pub. Date: Oct. 23, 2003

(54) Medicament for the stimulation of leucopoiesis and treatment of tumour and protozoan diseases acarinosis and arthropod-borne diseases and a method for production thereof

(76) Inventor: Hansjorg Eibl, Bovenden-Eddigehausen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/240,199

(22) PCT Filed: Mar. 29, 2001
(86) PCT No.: PCT/EP01/03609

(30) Foreign Application Priority Data
Mar. 30, 2000 (DE) 10015814.5

(51) Int. Cl. A61K 31/685; A61K 31/56

(52) U.S. Cl. 514/78; 514/171

(57) ABSTRACT

The invention relates to drug formulations for stimulating leukopoiesis and for treating tumor diseases and protozoal diseases, and acariasis and diseases caused by arthropods, which formulations are characterized in that they comprise an effective mixture composed of a) at least one phospholipid compound of the formula I:

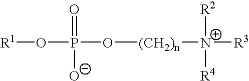

in which R¹ is a saturated or unsaturated hydrocarbon radical having from 16 to 24 C atoms, R², R³ and R⁴ are, in each case independently, H, a C₁-C₅-alkyl group, a C₃-C₆-cycloalkyl group or a C₁-C₅-hydroxyalkyl group, with two of R², R³ and R⁴ being able to form, with each other, a C₂-C₅-alkylene group which can optionally be substituted by an —O—, —S— or NR⁵ group, in which R⁵ is H, a C₁-C₅-alkyl group, a C₃-C₆-cycloalkyl group or a C₁-C₅-hydroxyalkyl group, and n is an integer from 2 to 6, as the active compound, comprising from 30 to 60 mol %, b) cholesterol and/or a cholesterol derivative, comprising from 25 to 65 mol %, and c) a phosphatidylmonoglycerol or phosphatidyloligoglycerol containing at least one oleyl group, comprising from 5 to 15 mol %, with a), b) and c) together comprising 100 mol %, and d) a water-miscible, physiologically acceptable alcohol, which possesses from 2 to 4 C atoms and which optionally contains water, and also, where appropriate, customary pharmaceutical auxiliary substances, with the components being present as a complex which is dispersed in water. The invention also relates to a process for preparing them.

### DESCRIPTION

The present invention relates to a novel drug formulation for stimulating leukopoiesis and for treating tumor diseases, protozoal diseases, in particular leishmaniases and amebic diseases, acariasis and diseases which are caused by arthropods, and to a process for producing it.

It is known that phospholipid compounds possessing an alkyl chain exhibit good activity against tumor diseases and protozoal diseases. However, major disadvantages of these compounds are, on the one hand, that the compounds possessing relatively long-chain hydrocarbon residues, in particular, exhibit poor solubility in aqueous solutions, thereby making them unsuitable both for intravenous (I.V.) administration and for oral administration in the form of solutions for drinking. On the other hand, many of these potent compounds are associated, with considerable side effects, which means that it is not possible to administer them in high doses over a relatively long period. The side effects are due, to a large extent, to the hemolytic effect of phospholipid compounds such as alkylphosphocholines having from 16 to 21 C atoms.

Protozoa are single-cell organisms, some of which are pathogenic parasites. The representatives which most frequently infect humans include plasmodia (malaria), trypanosomes (sleeping sickness), amebae, e.g. entamebae and acanthamebae (amebic dysentery, encephalitis) and leishmaniae (leishmaniasis).

Various tropical diseases which are caused by protozoa of the genus Leishmania and which are transmitted by blood-sucking insects are termed leishmaniases. Currently, three Leishmania species are known which cause very different syndromes: “kala azar”, in which the spleen and liver are affected, “oriental boil”, involving inflammatory reactions in the skin, and “espundia”, also involving symptoms in the mucous membranes of the upper respiratory tract and digestive tract. The course of all three diseases is less characteristic than in the case of other protozoal diseases and frequently proceeds insidiously. The incubation time can be weeks and even months. Very high mortality rates are frequently observed in untreated cases.

The therapy of leishmaniases is essentially still based on well-known antimony preparations, in particular sodium stibogluconate (Pentostam). The treatment is usually carried out for a period of from two to three weeks but then has to be interrupted for from one to two weeks because frequent side effects could otherwise reach threatening dimensions and become irreversible. The side effects include gastrointestinal irritation, circulatory disturbances up to and including shock and damage to the liver parenchyma. Another disadvantage which has emerged is that Leishmania strains which are antimony-resistant have already come into being. Other drugs which are employed are aromatic diamidines, pentamidine and amphotericin B. However, these agents are usually only used in combination with antimony compounds and, in addition to this, they also exhibit substantial side effects.

In humans, Entamoeba histolytica causes dysenteries and liver abscesses. In many countries in the world, the pathogen occurs very frequently; it causes about 36 to 50 million cases of disease per year with between 40 000 and 110 000 fatalities. The lifecycle is simple; infection takes place by way of cysts, which are assimilated together with contaminated water or contaminated foodstuffs. The cysts pass through the stomach unchanged and excyst in the large intestine, with four trophozoites, which are the actual amebae, originating from each cyst. Some of the trophozoites encyst once again in the rectum and in this way form the spores which are able to survive outside the human body. While the trophozoites can, on the one hand, live in the large intestine without causing a great deal of damage, they can also attack the intestinal wall. While this can give rise to small lesions in the mucous membrane, it can also give rise to ulcers which bleed massively. This results in bloody diarrhoeas, i.e. the complete picture of amebic dysentery. Another frequent manifestation of amebiasis is the amebic liver abscess. In this case, the amebae make their way from the intestine, through the mesenteric vessels and into the liver and give rise to large abscesses in this organ. If left untreated, both the amebic liver abscess and intestinal amebiasis are massively life-threatening.

E. histolytica trophozoites are unable to survive without the human host. By contrast, free-living amebae exist which are able, in rare cases, to elicit relatively serious diseases in humans. Acanthamebae (e.g. Acanthamoeba castellanii, Acanthamoeba culbertsoni) can cause chronic granulomatose encephalitis in immunosuppressed patients; in addition, cases of acanthameba keratitis occur relatively frequently in contact lens carriers. Naegleria fowleri is a free-living amebic flagellate. It typically lives in freshwater and can infect bathers. The parasite makes its way, via the nose and the olfactory nerves, into the brain and gives rise to peracute meningoencephalitis. While encephalitis cases due to acanthamebae and naeglerias are extremely rare, they have thus far had an extremely poor prognosis.

The chemotherapeutic agents which are currently used in E. histolytica infections are nitroimidazoles, with the primary agent being metronidazole.

E. histolytica does not possess any oxidative phosphorylation but, instead, obtains its energy by glycolysis. In the ameba, the oxidation of pyruvate to acetyl-CoA gives rise to reduced ferredoxin, which is able to reduce the nitroimidazole to a nitrosoimidazole. This aggressive substance damages the biomolecules of the ameba. Humans do not possess any such strong reducing agent and do not convert the metronidazole into the more poisonous nitrosoimidazole form. To date, there are still no verified reports regarding the distribution of metronidazole-resistant E. histolytica strains. However, there are regular reports of cases in which the metronidazole therapy is said to have failed and it has already been possible to generate partially resistant strains in the laboratory. If resistance were possibly to develop, it would be very important to have available novel substance classes which possess activity against E. histolytica since there are currently no satisfactory alternatives to the nitrimidazoles.

In contrast to E. histolytica, acanthamebae and naeglerias possess mitochondria and can live aerobically. They do not reduce nitroimidazoles and these compounds are therefore completely without effect. While acanthamebae are said to be sensitive to rifampicin and paromomycin, and naeglerias are said to be sensitive to amphotericin B, it has only been possible to cure encephalitides due to free-moving amebae in a few isolated cases.

DE application P 41 32 344.0-41 discloses processes for producing a drug which is suitable for oral or topical administration in the treatment of protozoal diseases, in particular leishmaniasis, and which comprises, as active compound, one or more compounds of the general formula:

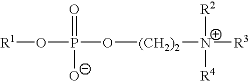

in which R¹ is a saturated or unsaturated hydrocarbon radical having from 12 to 20 C atoms, R², R³ and R⁴ are, in each case independently, H, a C₁-C₅-alkyl group, a C₃-C₆-cycloalkyl group or a C₁-C₅-hydroxyalkyl group, with two of R², R³ and R⁴ being able to form, with each other, a C₂-C₅-alkylene group which can optionally be substituted by an —O—, —S— or NR⁵ group, in which R⁵ is H, a C₁-C₅-alkyl group, a C₃-C₆-cycloalkyl group or a C₁-C₅-hydroxyalkyl group.

Compounds of this general formula exhibited a very much higher activity than that of sodium stibogluconate, particularly when administered orally or topically. However, substantial side effects, such as irritation of the gastrointestinal tract, also occurred occasionally, particularly at relatively high doses.

Another disadvantage of the abovementioned compounds is that it has thus far not been possible to administer alkylphosphocholines having chain lengths of more than 21 C atoms intravenously on account of their low solubility in water or, on the other hand, to administer alkylphosphocholines having chain lengths of 21 or fewer C atoms intravenously on account of hemolytic effects. In the past, compositions containing alkylphosphocholin have been packaged in liposomes for intravenous administration. The liposomes were composed of hexadecylphosphocholin, cholesterol and phosphatidylglycerol or of hexadecylphosphocholin, cholesterol and phosphatidylpolyethylene glycolene. However, the preparation of these liposomes is very elaborate and expensive since the liposomes require high pressure pressing or similar methods and, furthermore, the finished product suffers from the disadvantage that it can only be sterilized by filtration with great difficulty.

In order to better exploit the good activity of phospholipid compounds and to reduce the frequency with which the drug is taken, and nevertheless avoid side effects, it would be desirable to provide a drug formulation which comprises, as the active compound, a phospholipid compound which is in a form which enables it to be administered intravenously, even in high doses, and, in addition, also makes possible any form of administration, that is permits oral, topical, im, ip, sc and iv administration, with this administration being associated with few side effects.

The object of the present invention is achieved by means of a drug formulation which is characterized in that it comprises a mixture composed of

a) a phospholipid compound of the formula I:

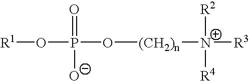

in which R¹ is a saturated or unsaturated hydrocarbon radical having from 16 to 24 C atoms, R², R³ and R⁴ are, in each case independently, H, a C₁-C₅-alkyl group, a C₃-C₆-cycloalkyl group or a C₁-C₅-hydroxyalkyl group, with two of R², R³ and R⁴ being able to form, with each other, a C₂-C₅-alkylene group which can optionally be substituted by an —O—, —S— or NR⁵ group, in which R⁵ is H, a C₁-C₅-alkyl group, a C₃-C₆-cycloalkyl group or a C₁-C₅-hydroxyalkyl group, and n is an integer from 2 to 6, as the active compound, comprising from 30 to 60 mol %,

b) cholesterol and/or a cholesterol derivative, comprising from 25 to 65 mol %, and

c) a phosphatidylmonoglycerol or phosphatidyloligoglycerol containing at least one oleyl group, comprising from 5 to 15 mol %, with a), b) and c) together comprising 100 mol %, and

d) a water-miscible, physiologically acceptable alcohol, which possesses from 2 to 4 C atoms and which optionally contains water, and also, where appropriate, customary pharmaceutical auxiliary substances and/or active compounds, with the components being present as a complex which is dispersed in water.

As a result of this special mixing ratio, and as a result of adding an alcohol, a complex which can be dispersed in water is surprisingly formed from the abovementioned components, where appropriate together with water. As a rule, liposomes are only formed under the influence of an ultrasonic treatment, or similar treatment, whereas, with the special mixing ratio which is essential for the invention, said liposome-like complex is formed, and is stable, without any external influence. As a result, it is also possible to incorporate other active compounds, such as amphotericin B, into the formulation.

While the molar mixing ratio can vary, such that either the phospholipid compound of the formula I(a), particularly in the case of phospholipids having chain lengths of from 22 to 24 C atoms, or the cholesterol and/or cholesterol derivative (b), particularly in the case of phospholipids having chain lengths of from 16 to 21 C atoms, is present in a slight excess, the ratio should in general not deviate too far from 1:1. The cholesterol derivative preferably comprises from 30 to 60 mol % of the mixture composed of a), b) and c).

The liposome-like complex which is formed from the components a), b) and c) and the alcohol containing water can easily be sterilized by filtering it through membranes having pore diameters of 0.8μ, 0.45μ and even 0.2μ. This represents a considerable advantage as compared with conventional liposomes, which are not easy to sterilize by filtration. In addition, it has turned out, surprisingly, that complexes according to the invention are extremely stable during storage.

Component b), i.e. the cholesterol or cholesterol derivative, also serves the purpose of improving the solubility in aqueous solutions of phospholipid compounds which are in accordance with the above definition. Cholesterol-like compounds, such as cholesterol oligoglycerols, are also suitable, for example.

Component c) of the complex according to the invention comprises phosphatidylglycerol and phosphatidyloligoglycerols. Preference is given to phosphatidyloligoglycerols containing from 1 to 4 glycerol radicals, in particular those containing fatty acid radicals which possess a cis double bond. Compounds of this nature which are preferred comprise dioleyl compounds such as dioleyl-SN-glycero-3-phosphoglycerol, dioleyl-SN-glycero-3-phospho-diglycerol, dioleyl-SN-glycero-3-phosphotriglycerol and dioleyl-SN-glycero-3-phosphotetraglycerol, which are preferably employed as Na⁺ salts. It is also possible to use compounds which contain an oleyl radical and another radical, preferably a palmitoyl radical. It is assumed that these compounds facilitate the incorporation of membrane components into bilayer structures and stabilize emulsions and the complex according to the invention. They preferably have a positive or negative excess charge of from +0.2 to +0.05.

The drug formulation preferably contains the components in a quantity which is such that the complex as a whole has a positive or negative excess charge. This is particularly advantageous when using phospholipids which possess relatively long hydrocarbon chains. However, the problem of compounds which possess relatively long chains exhibiting poorer solubility in water is only of importance in connection with intravenous administration and not in connection with oral administration.

Preference is given to the quantity of component c) being from 8 to 10 mol %.

In component a), i.e. the phospholipid of the formula I, the hydrocarbon radical R¹ can contain from 16 to 26 C atoms, with, in particular, from 18 to 24 C atoms being preferred and from 18 to 22 C atoms being more strongly preferred. R¹ is particularly preferably a hexadecyl, octadecyl, oleyl, elaidyl, eicosyl, eicosenyl-cis-(ω-9), heneicosyl, heneicosenyl, docosyl or docosenyl radical. The hydrocarbon radical can be either saturated or unsaturated, with the double bond(s) of the unsaturated radicals preferably being cis. If more than one cis double bond is present, they are then preferably not conjugated. The higher-membered, uneven-numbered hydrocarbon radicals have also proved to be particularly effective. Nonadecenyl and heneicosenyl are particularly preferred in this connection. The greatest preference is given to a compound of the formula I in which R¹=oleyl, in particular a cis-oleyl radical.

The polar constituent preferably comprises phosphocholin (PC), i.e. n is preferably equal to 2. R², R³ and R⁴ are preferably in each case methyl. Examples of other suitable radicals are ethyl, propyl, butyl and pentyl radicals, cyclopropyl, cyclobutyl, cyclopentyl and cyclohexyl radicals and hydroxymethyl, hydroethyl and hydroxypropyl radicals. Two of the radicals R², R³ and R⁴ can, for example, form a pyrrolidine group, a piperidine group or a morpholine group. At least one of the radicals R², R³ and R⁴ is preferably different from hydrogen; particularly preferably, all three radicals are different from hydrogen.

n can also be 3 or 4. Surprisingly, a stimulatory effect on leukopoiesis was obtained particularly when using compounds in which n is equal to 3.

A harmful hemolytic effect frequently occurs in the case of conventional formulations which comprise phospholipid compounds possessing short hydrocarbon chains. This effect is considerably decreased when the combination according to the invention is used. When short-chain phospholipid compounds possessing hydrocarbon radicals containing from 16 to 21 C atoms are used, preference is therefore given to cholesterol or cholesterol derivatives being present in the upper quantity range which is stipulated. Preference is consequently given to having a small excess of cholesterol or its derivative in the complex, such that the molar ratio between phospholipid compound of the formula I and cholesterol/cholesterol derivative is then 1:1-1.2.

When phospholipid compounds possessing longer hydrocarbon chains, containing from 22 to 24 carbon atoms, are used, the problem is then not so much one of hemolysis but one of lower solubility in water. For this reason, a molar ratio of phospholipid compound: cholesterol/cholesterol derivative of 1:0.5-1 is sufficient in this case.

Surprisingly, it has been found that a mixture composed of phospholipid compound of the formula I, cholesterol/cholesterol derivative and phosphatidyloligoglycerol or phosphatidylmonoglycerol in the abovementioned molar ratio can be readily dissolved in a water-miscible alcohol, preferably a physiologically tolerated alcohol. The mixing ratio of components a), b) and c) to alcohol is preferably in the range of 1:0.1 to 500. The mixture which results from this can then be readily diluted with water or another aqueous liquid and in this way brought to any arbitrary and desired concentration. In this way, it is possible to prepare I.V. solutions in which the alcohol content has been reduced to an acceptable concentration. I.V. solutions should not contain more than 3% ethanol, while oral solutions should not contain more than 10% ethanol.

For the purposes of the present invention, component d), i.e. the alcohol, is a water-miscible, physiologically tolerated alcohol that possesses from 2 to 4 carbon atoms. Ethanol, 2-propanol, 1,2-propanediol and 2-butanol, or combinations thereof, are particularly suitable. Greatest preference is given to 1,2-propanediol, particularly for iv preparations.

Another aspect of the present invention is a process for producing the drug formulation according to the invention, with a phospholipid compound of the formula I

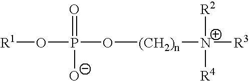

in which R¹ is a saturated or unsaturated hydrocarbon radical having from 16 to 24 C atoms, R², R³ and R⁴ are, in each case independently, H, a C₁-C₅-alkyl group, a C₃-C₆-cycloalkyl group or a C₁-C₅-hydroxyalkyl group, with two of R², R³ and R⁴ being able to form, with each other, a C₂-C₅-alkylene group which can optionally be substituted by an —O—, —S— or NR⁵ group, in which R⁵ is H, a C₁-C₅-alkyl group, a C₃-C₆-cycloalkyl group or a C₁-C₅-hydroxyalkyl group, and n is an integer from 2 to 4, being mixed, as active compound, in aqueous solution, with b) from 25 to 65 mol % of cholesterol and/or a cholesterol derivative and c) from 5 to 15 mol % of a phosphatidylmonoglycerol or phosphatidyloligoglycerol, with a), b) and c) together comprising 100 mol %, and a water-miscible, physiologically acceptable alcohol having from 2 to 4 C atoms being added to the resulting mixture, such that the components form a complex which is dispersed in water.

In order to implement the process, components a), b) and c), as defined above, are therefore first of all mixed in the abovementioned molar ratio. If an additional active compound is incorporated into the formulation according to the invention, this compound is preferably added to the mixture of a), b) and c). A water-miscible alcohol, which is a physiologically harmless alcohol containing from 2 to 4 C atoms, in particular ethanol, 2-propanol or 2-buntanol, since a certain quantity of the alcohol remains in the final drug formulation and this formulation is to be suitable both for oral and intravenous administration, is then added. Particular preference is given to using ethanol, 1,2-propanediol or 2-propanol. While ethanol is distinguished by a low degree of toxicity and consequently relatively good physiological tolerance, it is not so well suited for preparing complexes according to the invention which contain a relatively large quantity of cholesterol since cholesterol only dissolves in ethanol to a limited extent. For this reason, preference is given to using 1,2-propanediol, which also exhibits a very low degree of toxicity, in such cases. In this connection, the lipid components can initially be dissolved in propanediol plus chloroform and water, where appropriate. The solvent (mixture) can then be stripped off and the complexes which have been formed can be dissolved in 1,2-propanediol. Another possibility for improving solubility is to use cholesterol derivatives, in particular cholesterol phosphocholin (PC), which exhibits good solubility properties in ethanol, instead of cholesterol.

While the alcohol can be added to the mixture of components a), b) and c) at normal temperature (20° C.) it can also be added at an elevated temperature. In this case, heating to from 20 to 85° C. is preferred, with heating to from 60 to 80° C. being more strongly preferred. The phospholipid compound: alcohol molar ratio is 1:0.1 to 500. The quantity of alcohol which is added can consequently be varied over a wide range. The skilled person can easily determine the quantity of added alcohol which is optimal within the range disclosed herein. Ratios of from 1:50 to 200 are preferred, in particular.

The final concentrations of the alcohol which is present in the drug formulation according to the invention are expediently not more than 10% in the case of oral administration and not more than 3% in the case of intravenous administration. In the final drug formulation, the active compound, i.e. the phospholipid compound, is preferably present in a quantity of from 0.1 to 200 μmol/g.

In the mixture which is obtained in this way, the phospholipid compound according to formula I is present, together with the other components, as a complex which is dispersed, or dispersible, in water. Particularly when phospholipid compounds which are less readily soluble in water (such as those with relatively long hydrocarbon chains) are present, preference is given to adding component c), possessing an excess charge, in a quantity which is such that the overall complex possesses a positive or negative excess charge. As a result, the mixture can readily be diluted with water or other aqueous liquids, with particular preference being given, in this connection, to aqueous liquids which are physiologically tolerated.

Another advantage of the mixture which is obtained by means of the process according to the invention is that it can without difficulty be sterilized by filtration. In this connection, it is possible to use filters having pore sizes of 0.8μ, 0.45μ and even 0.2μ.

The formulation according to the invention can be galenically prepared in liquid or solid form. Particular preference is given to a formulation for intravenous or oral administration. However, a topical administration is also possible. In the case of oral administration, it is advantageous to dilute the active mixture with water or another physiological liquid, with a 5-fold to 150-fold dilution having proved to be particularly suitable. However, it is also possible to dilute the mixture more strongly since the complex remains soluble even at dilutions of from 1:1 000 to 1:10 000 and no separation of components in the form of crystals or precipitates has been observed. In the case of an I.V. administration, an injection or infusion in a volume of from 50 to 100 ml is advantageous since it is easily possible, in this way, to bring the alcohol concentration, in the case of ethanol, below a value of 1%. When the preferred 1,2-propanediol is used, there is no need at all to take account of the alcohol concentration. Dilutions with water or physiological aqueous solutions of from 1:5 to 1:150, preferably of from 1:10 to 1:20, are particularly suitable for this purpose. The daily dose of an effective quantity of the phospholipid compound, e.g. alkylphosphocholin of the formula I, is from 0.1 to 100 μmol/kg of bodyweight, preferably from 1 to 5 μmol/kg.

Because of the ready solubility, there is no need to use excess pressure, as is required when preparing liposomes, for preparing the solutions composed of the phospholipid compound and the other components. Simple sonication is sufficient as a rule; sometimes, even stirring is sufficient. This very much simplifies and cheapens the preparation process. In addition, it is possible to maintain sterile conditions without difficulty by storing in appropriately concentrated alcoholic solutions. These advantages also apply when a further active compound is additionally incorporated into the formulation.

However, instead of diluting with aqueous liquids, it is also possible to produce the drug formulation according to the invention in another form, for example as powders, tablets or capsules or else as an ointment. In this case, the alcohol is preferably added in smaller quantity than when preparing the formulation for use in liquid form. In this present instance, preference is given to a phospholipid compound:alcohol molar mixing ratio of from 1:5 to 100. Where appropriate, the alcohol can also be at least partially removed once again from the mixture in order to obtain a concentrated formulation. At the same time, the drug formulation can be mixed with customary, physiologically tolerated fillers, carrier substances, diluents and/or auxiliary substances and poured into hollow cells of appropriate size or aliquoted into capsules of appropriate size or granulated and then pressed, where appropriate in the added presence of other customary auxiliary substances, into tablets. The formulation can, for example, be mixed with one or more of the following auxiliary substances: starch, cellulose, lactose, formalin, casein, modified starch, magnesium stearate, calcium hydrogen phosphate, highly disperse silicic acid, talc and phenoxyethanol. The resulting mixture can, where appropriate, be granulated together with an aqueous solution composed, for example, of gelatin, starch, polyvinylpyrrolidone, vinylpyrrolidone-vinyl acetate copolymer and/or polyoxyethylene sorbitate monooleate and subsequently pressed into tablets or aliquoted into capsules.

Surprisingly, it has emerged that the drug formulation according to the invention also exhibits good activity against acariasis, in particular mange, and against diseases caused by arthropods. Additional active compounds can, if desired, aid, augment or broaden these indications. In particular, adding amphotericin B brought about a synergistic amplification of the activity directed against protozoal diseases and a broadening of the activity of the formulation to cover systemic fungal diseases.

The following examples are intended to illustrate the invention.

### EXAMPLES

Preparing liposomal formulations without ethanol
General guide values for weighing out:

| | Concentration | 250 ml |
|---|---|---|
| | (μmol) | (μmol) |
| Alkyl-PC | 45.0 | 11250 |
| (≦21 C atoms) | | |
| Cholesterol | 47.5 | 11875 |
| 1,2-Dioleoyl-sn-G-4-PG | 7.5 | 1875 |
| (Na⁺-salt) | | |
| | 100.0 | 25000 |

| | |
|---|---|
| Lipid content: | 100 μmol/ml |
| C total lipid: | 0.1 M |
| C active compound: | 0.045 M |

### Example 1

### Octadecyl-1-PC: Volume 250 ml

| | | Amount weighed out | | |
|---|---|---|---|---|
| MW | | (g) | (μmol) | μmol/ml |
| 435.63 | Octadecyl-1-PC | 4.922 | 11299 | 45.2 |
| 396.66 | Cholesterol | 4.783 | 12370 | 49.5 |
| 688.90 | 1,2-Dioleoyl-sn-G-3-PG | 1.465 | 2127 | 8.5 |
| | (Na⁺-salt) | | | |
| | | 11170 | 25796 | 103.2 |

The weigh-out amount of 11.17 g is treated, in a 1 l round-bottomed flask, with 100 ml of 2-propanol, 50 ml of CHCl₃ and 1 ml of H₂O and brought into solution at 50° C. After everything has been dissolved, the solvent is removed in vacuo at from 30 to 35° C. The residual solvent is removed in vacuo, at 30° C., in a drying oven over a period of 30 minutes. The dry residue is treated with 225 ml of a 0.25 M solution of 1,2-propanediol (MW 76.10) and the mixture is heated to 50° C. on a rotary evaporator while rotating. The mixture is sonicated at 50° C. for 15 minutes while rotating, after which the sonication is stopped for 2 minutes; this sonication procedure is then repeated a further two times. The suspension is then uniform. It can readily be filtered through a glass filter and then through a 0.8μ filter. The liposomes which are obtained in this way are stable at 4° C. for a period of at least 12 months. Corresponding solutions were also obtained using hexadecyl-PC, heptadecyl-PC and nonadecyl-PC.

### Example 2

### Arachinyl-1-PC: Volume 250 ml

| | | Amount weighed out | | |
|---|---|---|---|---|
| MW | | (g) | (μmol) | μmol/ml |
| 463.68 | Arachinyl-1-PC | 5.310 | 11 464 | 45.9 |
| 396.66 | Cholesterol | 4.980 | 12 880 | 51.5 |
| 688.90 | 1,2-Dioleoyl-sn-G-3-PG | 1.700 | 2 133 | 8.5 |
| | (Na⁺-salt) | | | |
| | | 11.990 | 26 477 | 105.9 |

The lipid mixture, 11.99 g, is treated as in Example 1 and brought into a liposomal formulation.

### Example 3

### Oleyl-1-PC: Volume 250 ml

| | | Amount weighed out | | |
|---|---|---|---|---|
| MW | | (g) | (μmol) | μmol/ml |
| 433.61 | Oleyl-1-PC | 5.041 | 11 626 | 46.5 |
| 386.66 | Cholesterol | 4.7570 | 12 303 | 49.2 |
| 797.03 | 1,2-Dioleoyl-sn-G-3-PG | 1.555 | 1 951 | 7.8 |
| | (Na⁺-salt) | | | |
| | | 11.353 | 25 880 | 103.5 |

The lipid mixture, 11.353 g, is treated as in Example 1 and brought into a liposomal formulation.

### Example 4

### (Z)-10-nonadecenyl-1-PC: Volume 250 ml

| | | Amount weighed out | | |
|---|---|---|---|---|
| MW | | (g) | (μmol) | μmol/ml |
| 477.64 | (Z)-10-Nonadecenyl-1-PC | 5.471 | 12 223 | 48.9 |
| 396.66 | Cholesterol | 4.973 | 12 861 | 51.4 |
| 688.90 | 1,2-Dioleoyl-sn-G-3-PG | 1.647 | 2 066 | 8.3 |
| | (Na⁺-salt) | | | |
| | | 12.091 | 27 150 | 108.6 |

The lipid mixture, 12.091 g, is treated as in Example 1 and brought into a liposomal formulation.

### Example 5

### (Z)-10-eicosenyl-1-PC: Volume 250 ml

| | | Amount weighed out | | |
|---|---|---|---|---|
| MW | | (g) | (μmol) | μmol/ml |
| 461.66 | (Z)-10-Eicosenyl-1-PC | 5.170 | 11 198 | 44.8 |
| 396.66 | Cholesterol | 4.938 | 12 450 | 49.8 |
| 688.90 | 1,2-Dioleoyl-sn-G-3-PG | 1.385 | 2 011 | 8.0 |
| | (Na⁺-salt) | | | |
| | | 11.493 | 25 569 | 102.6 |

The lipid mixture, 11.493 g, is treated as in Example 1 and brought into a liposomal formulation.

### Example 6

### (Z)-10-heneicosenyl-1-PC: Volume 250 ml

| | | Amount weighed out | | |
|---|---|---|---|---|
| MW | | (g) | (μmol) | μmol/ml |
| 475.69 | (Z)-10-Henicosenyl-1-PC | 5.094 | 10 709 | 42.8 |
| 396.66 | Cholesterol | 4.910 | 12 378 | 49.5 |
| 688.90 | 1,2-Dioleoyl-sn-G-3-PG | 1.312 | 1 905 | 7.6 |
| | (Na⁺-salt) | | | |
| | | 11.316 | 24 992 | 99.9 |

The lipid mixture, 11.316 g, is treated as in Example 1 and brought into a liposomal formulation.

General guide values for weighing out:

| | Concentration | 250 ml |
|---|---|---|
| | (μmol/ml) | (μmol) |
| Alkyl-PC | 55.0 | 13 750 |
| (≧22 C atoms) | | |
| Cholesterol | 37.5 | 9 375 |
| 1,2-Dioleoyl-sn-G-4-PG | 7.5 | 1 875 |
| (Na⁺-salt) | | |
| | 100.0 | 25.000 |

| | |
|---|---|
| Lipid content: | 100 μmol/ml |
| C total lipid: | 0.1 M |
| C active compound: | 0.055 M |

### Example 7

### Erucyl-PC: Volume 250 ml

| | | Amount weighed out | | |
|---|---|---|---|---|
| MW | | (g) | (μmol) | μmol/ml |
| 489.72 | Erucyl-PC | 5.520 | 11 272 | 45.1 |
| 386.66 | Cholesterol | 2.910 | 7 526 | 30.1 |
| 797.03 | 1,2-Dioleoyl-sn-G-3-PG | 1.200 | 1 506 | 6.0 |
| | (Na⁺-salt) | | | |
| | | 9.630 | 20 304 | 81.2 |

The lipid mixture, 9.63 g, is treated as in Example 1 and brought into a liposomal formulation.

### Example 8

### Erucyl-1-P-(CH₂)₃-C: Volume 250 ml

| | | Amount weighed out | | |
|---|---|---|---|---|
| MW | | (g) | (μmol) | μmol/ml |
| 503.74 | Erucyl-1-P-(CH₂)₃-C | 6.110 | 12 129 | 48.5 |
| 386.66 | Cholesterol | 2.540 | 6 559 | 26.2 |
| 797.03 | 1,2-Dioleoyl-sn-G-3-PG | 1.200 | 1 506 | 6.0 |
| | (Na⁺-salt) | | | |
| | | 9.850 | 20 194 | 80.7 |

The lipid mixture, 9.85 g, is treated as in Example 1 and brought into a liposomal formulation.

### Example 9

### (Z.Z)-6.15-tetracosadienyl-t-PC: Volume 250 ml

| | | Amount weighed out | | |
|---|---|---|---|---|
| MW | | (g) | (μmol) | μmol/ml |
| 515.75 | (Z.Z)-6.15-Tetra- | 8.010 | 15 531 | 62.1 |
| | cosadienyl-1-PC | | | |
| 386.66 | Cholesterol | 4.210 | 10 888 | 43.6 |
| 797.03 | 1,2-Dioleoyl-sn-G-3-PG | 1.690 | 2 120 | 8.5 |
| | (Na⁺-salt) | | | |
| | | 8.016 | 28 539 | 114.2 |

The lipid mixture, 8.016 g, is treated as in Example 1 and brought into a liposomal formulation.

### Example 10

### Toxicity and Activity Tests

TABLE 1

| | Necessary administration quantities in μmol | | |
|---|---|---|---|
| | | Oleyl-PC/Nona- | |
| Form | Hexydecyl-PC | decyl-PC | Erucyl-PC |
| Free (oral) | 60 | 20 | 100 |
| According | 40 | <5 | <5 |
| to the | | | |
| invention | | | |
| iv | | | 10 |
| Toxicity | 90 | Still not | |
| | | reached at 100 | |

These experiments in rats/mice demonstrate the superiority of the phosphocholin compounds, which are used in accordance with the invention as active compounds, when they are incorporated into liposome-like complexes according to the invention.

### Example 11

### Drug Formulations

In the following table, some combinations of a drug formulation according to the invention are depicted by way of example. Alkylphosphocholines and cholesterol were weighed out in the molar ratios 1.3 (1:0.75, excess of alkylphosphocholin) to 0.8 (1:1.25, excess of cholesterol) and in each case dissolved in ethanol or 2-propanol. The solutions were treated, at from 60 to 80° C. and while being stirred, with different quantities of H₂O or physiologically tolerated solutions, or else the active compound concentrate was added to H₂O or physiologically tolerated solutions. The emulsion which formed in this connection was sterilized by filtration through 0.8μ, 0.45μ and 0.2 filters.

The following table gives suitable daily doses and dilutions.

TABLE 2

| | Molar ratio | | | Active | Daily |
|---|---|---|---|---|---|
| | (PC/Chol/ | | Dilution | compound | dose |
| Alkyl-PC/Chol | ethanol) | Solvent | (H₂O) | (μmol/g) | (180 μmol/kg) |
| C₁₆-O-PC/Chol | 1:0.84:363 | ethanol | 324 g (1:10) | 6.0 | 30 ml |
| (MW 407.511)/(MW 386.659) | | MW 46.07 | 684 g (1:20) | 3.0 | 60 ml |
| a) 875 mg-/700 mg- | | 36 g (20° C.) | 3564 g (1:100) | 0.6 | 300 ml |
| 2.15 mmol/1.81 mmol | | 781 mmol | | | |
| b) 814 mg-/698 mg- | 1:0.9:217 | 20 g (40° C.) | 180 g (1:10) | 10.0 | 18 ml |
| 2.00 mmol/1.81 mmol | | 434 mmol | 380 g (1:20) | 5.0 | 36 ml |
| | | | 1980 g (1:100) | 1.0 | 180 ml |
| C₁₈-O-PC/Chol | 1:0.89:398 | ethanol | 360 g (1:10) | 2.4 | 75 ml |
| (MG 435.625) | | | 760 g (1:20) | 1.2 | 150 ml |
| a) 948 mg-/749 mg- | | 40 g (20° C.) | 3960 g (1:100) | 0.24 | 750 ml |
| 2.18 mmol/1.94 mmol | | 868 mmol | | | |
| b) 858 mg-/851 mg- | 1:1.11:440 | 40 g (30° C.) | 360 g (1:10) | 2.2 | 89 ml |
| 1.97 mmol/2.20 mmol | | 868 mmol | 760 g (1:20) | 1.1 | 178 ml |
| | | | 3960 g (1:100) | 0.22 | 890 ml |
| c) 255 mg-/245 mg- | 1:1.06:36.7 | 1 g (80° C.) | 9 g (1:10) | 59.0 | 3 ml |
| 0.59 mmol/0.63 mmol | | 21.7 mmol | 19 g (1:20) | 29.5 | 6 ml |
| | | | 99 g (1:100) | 5.9 | 30 ml |
| d) 1041 mg-/1062 mg- | 1:1.15:91 | 10 g (60° C.) | 90 g (1:10) | 24.0 | 7.5 ml |
| 2.39 mmol/2.75 mmol | | 217 mmol | 190 g (1:20) | 12.0 | 15.0 ml |
| | | | 990 g (1:100) | 2.4 | 75.0 ml |
| e) 1414 mg-/1234 mg- | 1:0.99:25.6 | 2-propanol | 45 g (1:10) | 65.0 | 2.8 ml |
| 3.24 mmol/3.20 mmol | | MW 60.09 | 95 g (1:20) | 32.5 | 5.6 ml |
| | | 5 g (70° C.) | 450 g (1:100) | 6.5 | 28.0 ml |
| | | 83 mmol | | | |
| f) 937 mg-/908 mg - | 1:1.09:15.3 | 2 g (70° C.) | 18 g (1:10) | 108.0 | 1.7 ml |
| 2.15 mmol/2.35 mmol | | 33 mmol | 38 g (1:20) | 54.0 | 3.4 ml |
| | | | 198 g (1:100) | 5.4 | 17.0 ml |
| g) 938 mg -/908 mg - | 1:1.09:38.6 | 5 g (60° C.) | 45 g (1:10) | 43.0 | 4.2 ml |
| 2.15 mmol/2.35 mmol | | 83 mmol | 95 g (1:20) | 86.0 | 8.4 ml |
| | | | 445 g (1:100) | 4.3 | 42.0 ml |
| C_{18:1}-O-PC/Chol | 1:1.03:63.2 | | 36 g (1:10) | 27.0 | 6.7 ml |
| MW 433.609) | | | 76 g (1:20) | 13.5 | 13.4 ml |
| 460 mg-/423 mg- | | 4 g (30° C.) | 396 g (1:100) | 2.7 | 67.0 ml |
| 1.06 mmol/1.09 mmol | | 67 mmol | | | |
| C₂₀-O-PC/Chol | 1:1.1:349 | | 360 g (1:10) | 2.20 | 82.0 ml |
| (MW 463.679) | | | 760 g (1:20) | 1.1 | 164.0 ml |
| 885 mg-/808 mg- | | 40 g (20° C.) | 3960 g (1:100) | 0.22 | 820.0 ml |
| 1.91 mmol/2.09 mmol | | 666 mmol | | | |
| C_{21:1}-2-O-PC/Chol | 1:0.93:34 | | 22.5 g (1:10) | 49.2 | 3.7 ml |
| (MW 475.710) | | | 4.5 g (1:20) | 24.6 | 7.4 ml |
| 583 mg-/443 mg- | | 2.5 g | 247.5 g (1:100) | 4.92 | 37.0 ml |
| 1.23 mmol/1.15 mmol | | (60° C.) | | | |
| | | 42 mmol | | | |
| C₂₂-O-PC/Chol | 1:1.22:356 | | 360 g (1:10) | 4.7 | 38.3 ml |
| (MW 491.732) | | | 760 g (1:20) | 2.35 | 76.6 ml |
| 917 mg-/880 mg- | | 40 g (30° C.) | 3960 g (1:100) | 0.47 | 383.0 ml |
| 1.87 mmol/2.28 mmol | | 666 mmol | | | |
| C_{22:1}-O-PC/Chol | 1:0.9:31 | | 18 g (1:10) | 51.0 | 3.5 ml |
| (MW 489.716) | | | 38 g (1:20) | 26.5 | 7.0 ml |
| 518 mg-/371 mg- | | 2 g (70° C.) | 178 g (1:100) | 5.1 | 35.0 ml |
| 1.06 mmol/0.96 mmol | | 33 mmol | | | |
| C_{22:1}-O-PC₃/Chol | 1:1.04:33 | | 18 g (1:10) | 50.0 | 3.6 ml |
| (MW 503.763) | | | 38 g (1:20) | 25.0 | 7.2 ml |
| 509 mg-/406 mg- | | 2 g (60° C.) | 178 g (1:100) | 5.0 | 36.0 ml |
| 1.01 mmol/1.05 mmol | | 33 mmol | | | |

Legend:
PC = phosphocholine
Chol = cholesterol
MW = molecular weight

### Example 12

### Treatment of Leishmaniasis in Dogs

Dogs are typical carriers of leishmaniasis, particularly in the Mediterranean countries.

It has emerged that, in free (which is not in accordance with the invention) form, the active compounds which are used in accordance with the invention exhibit a relatively high degree of toxicity, as is manifested by the animals suffering a marked loss of weight. The following experiments using active compounds which have been prepared in accordance with the invention demonstrate that there is no loss of weight, that relatively low doses exhibit activity and that an antileishmaniasis effect is evident after only a few days.

a) Dog 1: “Leo” (dachshund, male)
Bodyweight: 9 kg

| | |
|---|---|
| Therapy: | oleyl-PC in accordance with Example 3 |
| | (MW 433.61-37.6 μmol/ml) |
| | oral administration |

| | |
|---|---|
| Aim: | 50 μmol (21.7 mg)/kg/week |
| | i.e. at 9 kg = 450 μmol = 195 mg |

μmol (mg)/kg/week

| | | |
|---|---|---|
| 52.7 (22.8 mg) | Week 1- | 33.8 μmol = 0.9 ml |
| | | both morning and evening |
| | | per week = 12.6 ml = 474 μmol/ |
| | | 9 kg |
| | (Once- | |
| | only dose- | 33.8 μmol = 14.7 mg |
| | per kg- | 3.8 μmol = 1.7 mg) |
| | Week 2- | as week 1 |
| | Week 3- | as week 1 |

b) Dog 2
Bodyweight: 25 kg

| | |
|---|---|
| Therapy: | oleyl-PC in accordance with Example 3 |
| | (MW 433.61-37.6 μmol/ml) |
| | oral administration |

μmol (mg)/kg/week

| | | |
|---|---|---|
| 52.6 (22.8 mg) | Week 1- | 2.5 ml |
| | | both morning and evening |
| | | per week = 35 ml = 1316 μmol/ |
| | | 25 kg |
| | (Once- | |
| | only dose- | 94 μmol = 40.8 mg |
| | per kg- | 3.8 μmol = 1.7 mg) |
| | Week 2- | as week 1 |
| | Week 3- | as week 1 |

### Example 13

Entamoeba histolytica SFL-3 and HM-1: IMSS (American Type Culture collection, order number ATCC 30459), pathogenic amebae of zymodeme II, were cultured, at 37° C., in TYI-S-33 medium (Diamond et al., Trans. Roy. Soc. Trop. Med. Hyg. 72: 431-432 (1978)) containing 10% bovine serum. The cultures of SFL-3 were kept in 100 ml glass bottles while the cultures of HM-1:IMSS were kept in 50 ml tissue culture flasks.

The strains are deposited in the American Type Culture Collection.

38 h cultures of E histolytica were used for measuring the cytotoxicity of the alkylphosphocholines. The amebae were released from the culture vessels by shaking, centrifuged down for 3 min at 2 000 revolutions per minute and 4° C. in a Heraeus Minifuge RF, resuspended in 20 ml of TYI-S-33 medium and counted in a hemocytometer chamber.

For each measurement, in each case 8-10×10⁵ amebae were provided in 12 ml of medium in Pyrex tubes fitted with a screw closure and the alkylphosphocholine was added in a volume of 660 μl. % % (w/v) ethanol in double-distilled water was used for dissolving hexadecylphosphocholine and octadecylphosphocholine, while double-distilled water on its own was used for all the other substances and the liposomal preparations. 5% ethanol or double-distilled water was correspondingly added to the control cultures. The concentrations of alkylphosphocholines employed were 100 μM, 50 μM, 20 μM, 10 μM and 5 μM. Six cultures were in each case prepared for each of the concentrations.

The effect of the alkylphosphocholines was determined after 24 h and after 48 h. For this, in each case three of the cultures were shaken up and transferred into in each case three plastic centrifuge tubes. The amebae were centrifuged down for 5 min at 2 200 rpm and 4° C. and suspended in a final volume of 1 ml in a 1:1 mixture of Trypan blue (Sigma) and PBS. Each of the samples was then counted in the hemocytometer and the number of living and dead, stained blue due to having taken up the die, amebae was recorded.

The results were analyzed using the “Probit” program (Wernsdorfer and Wernsdorfer, Mitt. Österr. Ges. Tropenmed. Parasitol. 17:221-228 (1995)).

TABLE 3

| STRAIN SFL-3 | | STRAIN HM-1:IMSS | |
|---|---|---|---|
| APC 1 | APC L1 | APC 1 | APC L1 |
| 88 | 75 | 94 | 124 (day 1) |
| 50 | 43 | 83 | 114 (day 2) |
| APC 2 | APC L2 | APC 2 | APC L2 |
| 36 | 85 | 80 | 131 (day 1) |
| 24 | 39 | 70 | 101 (day 2) |
| APC 9 | APC L9 | APC 9 | APC L9 |
| 32 | 39 | 121 | 128 (day 1) |
| 16 | 30 | 80 | 94 (day 2) |
| APC 10 | APC L10 | APC 10 | APC L10 |
| 24 | 79 | 112 | 118 (day 1) |
| 20 | 50 | 87 | 127 (day 2) |

Table 4: ED₅₀ values in [μM] of the alkylphosphocholines tested on two strains of Entamoeba histolyticya
APC = alkylphosphocholine
L = liposomal formulation

Substances:

1=hexadecylphosphocholine

2=octadecylphosphocholine

9=oleylphosphocholine

10=(Z)-10-nonadecenylphosphocholine

The tests were carried out as described above. The E. histolytica strains SFL-3 and HM-1:IMSS were tested. In each case six cultures were prepared for each concentration of alkylphosphocholine in pure or liposomal form. Three of the cultures were counted after 24 j and three further cultures were counted after 48 h. In each case two independent experiments were carried out for each of the two strains. The ED₅₀ values were determined using the “Probit” program and are shown in table 1.

Using two pathogenic strains of E. histolytica, it was demonstrated that alkylphosphocholines are able, in pure form and in liposomal form, to kill amebae. This takes place at a concentration which is perfectly attainable in an animal model. Oleylphosphocholine is the substance, which, in liposomal formulation, was the most effective in the case of E. histolytica.

The alkylphosphocholines, in pure form and liposomal form, constitute a completely novel form of therapy against amebae. They are not dependent on the anaerobic metabolism of the amebae but, instead, interfere in the relatively sensitive membrane structure of the amebae. For this reason, free living amebae, as well as the classical E. histolytica, are a possible target for the therapy using alkylphosphocholines in pure form or liposomal form.

### Example 14

Diseases which are caused by mites (Acarina), such as mange (Sarcoptes scabiei), and which are frequently difficult to treat, since Sarcoptes mange is a disease which is very resistant to treatment, frequently occur in a large number of animals, particularly dogs and chamois, and also in humans.

Dogs which were suffering from Sarcoptes mange were treated by injection with the drug according to the invention using the same dosage and the same drug composition as described for Leishmaniasis in example 12. The diseased animals were treated for 10 days in accordance with the therapy plan specified in the following table and in dependence on their body weight. Both a marked clinical improvement and an improved psychic state were evident after from 4 to 6 days. The dogs became lively and playful and communicated with their surroundings. If all the symptoms of the disease had not disappeared in the 1st decade, the treatment was repeated once again after 8 weeks In the case of those treated dogs which were still exhibiting slight signs of disease after the first treatment decade, these signs were completely eliminated by the treatment in the second decade, in connection with which the physical and psychic state of the treated animals improved markedly.

### Example 15

Amounts weighed out

| Substance | MW | mg | mmol |
|---|---|---|---|
| Amphotericin B × 1 HCl | 960.570 | 517 | 538 |
| Erucyl-PC | 489.716 | 1 944 | 3 997 |
| Cholesterol | 386.660 | 1 745 | 4 513 |
| PP-G-PG₂; Na⁺ | 819.040 | 831 | 1 015 |

(PP-G-PG₂ = dipalmitoylglycerophosphodiglycerol)

The substance mixture is dissolved in 2-propanol in the hot, filtered and freed from the solvent in vacuo. The residue is treated with 100 ml of 0.275 M 1,2-propanediol;

warming at 55° C. for 10 minutes

ultrasonication, 50% power—15 minutes at 55° C.

ultrasonication, 100% power—15 minutes at 55° C.

The dispersion is filtered through a glass fiber filter. After that, the filtrate can be sterilized by filtration without any difficulty (0.45 μm and 0.20 μm filters).

Content: Amphotericin B, 5 mg/ml

Erucyl-PC, 40 μmol/ml

### Example 16

Amounts weighed out

| Substance | MW | mg | mmol |
|---|---|---|---|
| Amphotericin B × 1 HCl | 960.570 | 893 | 930 |
| Erucyl-PC | 503.743 | 2 153 | 4 274 |
| Cholesterol | 386.660 | 1 765 | 4 570 |
| PP-G-PG₂; Na⁺ | 819.040 | 835 | 1 002 |

The substance mixture is dissolved in 2-propanol in the hot, filtered and freed from the solvent in vacuo. The residue is treated with 100 ml of 0.275 M 1,2-propanediol;

warming at 55° C. for 10 minutes

ultrasonication, 50% power—15 minutes at 55° C.

ultrasonication, 100% power—15 minutes at 55° C.

The dispersion is filtered through a glass fiber filter. After that, the filtrate can be sterilized by filtration without any difficulty (0.45 Am and 0.20 μm filters).

Content: Amphotericin B, 9 mg/ml

Erucyl-PC_{H3}, 40 μmol/ml

*Erucyl-PCH₃ is a phosphocholine with a phospho-trimethylammonium distance which has been extended to 3 C atoms.

### Example 17

Amount weighed out

| Substance | MW | mg | mmol |
|---|---|---|---|
| Amphotericin B × 1 HCl | 960.570 | 481 | 500 |
| Erucyl-PC_{H3} | 503.743 | 2 303 | 4 572 |
| Cholesterol | 386.660 | 1 789 | 4 626 |
| OO-G-PG | 797.030 | 762 | 956 |

(OO-G-PG = dioleoylglycerophosphoglycerol)

The substance mixture is dissolved in 2-propanol in the hot, filtered and freed from solvent in vacuo. The residue is treated with 100 ml of 0.275 M 1,2-propanediol;

warming at 55° C. for 10 minutes

ultrasonication, 50% power—15 minutes at 55° C.

ultrasonication, 100% power—15 minutes at 55° C.

The dispersion is filtered through a glass fiber filter. After that, the filtrate can be sterilized by filtration without any difficulty (0.45 μM and 0.20 μM filters).

Content: Amphotericin B, 4.8 mg/ml

Erucyl-PC_{H3}, 45 μmol/ml

*Erucyl-PCH3 is a phosphocholine with a phospho-trimethylammonium distance which has been extended to 3 C atoms.

TABLE 4
Sarcoptis therapy in the dog
Dosage: ml of the liposomal suspension containing 30 mmol
of oleyl-PC according to example 3 per ml

| Days | Body weight | | | | | | | |
|---|---|---|---|---|---|---|---|---|
| of the week | 10 kg | 15 kg | 20 kg | 25 kg | 30 kg | 35 kg | 40 kg | 50 kg |
| Monday | 1.4 ml | 2.1 ml | 2.8 ml | 3.5 ml | 4.2 ml | 4.9 ml | 5.6 ml | 7.0 ml |
| Tuesday | 1.4 ml | 2.1 ml | 2.8 ml | 3.5 ml | 4.2 ml | 4.9 ml | 5.6 ml | 7.0 ml |
| Wednesday | 1.4 ml | 2.1 ml | 2.8 ml | 3.5 ml | 4.2 ml | 4.9 ml | 5.6 ml | 7.0 ml |
| Thursday | 1.4 ml | 2.1 ml | 2.8 ml | 3.5 ml | 4.2 ml | 4.9 ml | 5.6 ml | 7.0 ml |
| Friday | 1.4 ml | 2.1 ml | 2.8 ml | 3.5 ml | 4.2 ml | 4.9 ml | 5.6 ml | 7.0 ml |
| Saturday | 1.4 ml | 2.1 ml | 2.8 ml | 3.5 ml | 4.2 ml | 4.9 ml | 5.6 ml | 7.0 ml |
| Monday | 1.4 ml | 2.1 ml | 2.8 ml | 3.5 ml | 4.2 ml | 4.9 ml | 5.6 ml | 7.0 ml |
| Tuesday | 1.4 ml | 2.1 ml | 2.8 ml | 3.5 ml | 4.2 ml | 4.9 ml | 5.6 ml | 7.0 ml |
| Wednesday | 1.4 ml | 2.1 ml | 2.8 ml | 3.5 ml | 4.2 ml | 4.9 ml | 5.6 ml | 7.0 ml |
| Thursday | 1.4 ml | 2.1 ml | 2.8 ml | 3.5 ml | 4.2 ml | 4.9 ml | 5.6 ml | 7.0 ml |
| Total quantity | 14 ml | 21 ml | 28 ml | 35 ml | 42 ml | 49 ml | 56 ml | 70 ml |

1. A drug formulation for stimulating leukopoesis and for treating acariasis, diseases caused by arthropods and tumor diseases and protozoal diseases,
characterized in that
it comprises an effective mixture composed of
a) at least one phospholipid compound of the formula I: 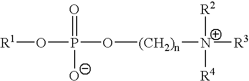
in which R¹ is a saturated or unsaturated hydrocarbon radical having from 16 to 24 C atoms,
R², R³ and R are, in each case independently, H, a C₁-C₅-alkyl group, a C₃-C₆-cycloalkyl group or a C₁-C₅-hydroxyalkyl group, with two of R², R³ and R⁴ being able to form, with each other, a C₂-C₅-alkylene group which can optionally be substituted by an —O—, —S— or NR⁵ group, in which R⁵ is H, a C₁-C₅-alkyl group, a C₃-C₆-cycloalkyl group or a C₁-C₅-hydroxyalkyl group,
and n is an integer from 2 to 6, as the active compound, comprising from 30 to 60 mol %,
b) cholesterol and/or a cholesterol derivative, comprising from 25 to 65 mol %,
c) a phosphatidylmonoglycerol or phosphatidyloligoglycerol containing at least one oleyl group, comprising from 5 to 15 mol %, with a), b) and c) together comprising 100 mol %, and
d) a water-miscible, physiologically acceptable alcohol, which possesses from 2 to 4 C atoms and which optionally contains water, and also, where appropriate, customary pharmaceutical auxiliary substances and/or active compounds, with the components being present as a complex which is dispersed in water.

2. The drug formulation as claimed in claim 1, characterized in that
its component b) comprises from 30 to 60 mol %.

3. The drug formulation as claimed in claim 1 or 2, characterized in that
the component c) is selected from phosphatidylmonoglycerols or phosphatidyloligoglycerols containing from 1 to 4 glycerol radicals.

4. The drug formulation as claimed in claim 3, characterized in that
the component c) is selected from dioleyl-SN-glycero-3-phosphoglycerol, dioleyl-SN-glycero-3-phospho-diglycerol, dioleyl-SN-glycero-3-phosphotriglycerol and dioleyl-SN-glycero-3-phosphotetraglycerol.

5. The drug formulation as claimed in one of claims 1 to 4,
characterized in that
the alcohol is ethanol, 1,2-propanediol, 2-propanol or 2-butanol.

6. The drug formulation as claimed in one of the preceding claims,
characterized in that
the phospholipid compound is an alkylphosphocholine in which n=2.

7. The drug formulation as claimed in one of the preceding claims,
characterized in that
it is present at a suitable concentration which is obtained by diluting with water or an aqueous, physiological liquid.

8. The drug formulation as claimed in one of the preceding claims,
characterized in that
it comprises the phospholipid compound of the formula I in a quantity of from 0.1 to 200 mmol/g.

9. The drug formulation as claimed in one of the preceding claims,
characterized in that
it comprises a phospholipid compound of the formula I in which R¹ is a hydrocarbon radical having from 16 to 21 C atoms.

10. The drug formulation as claimed in claim 9,
characterized in that
the cholesterol and/or cholesterol derivative is present in a molar excess in relation to the phospholipid compound.

11. The drug formulation as claimed in one of claims 1 to 8,
characterized in that
it comprises a phospholipid compound of the formula I in which R¹ is a hydrocarbon radical having from 22 to 24 C atoms.

12. The drug formulation as claimed in claim 11,
characterized in that
the phospholipid compound is present in a molar excess in relation to the cholesterol and/or cholesterol derivative.

13. The drug formulation as claimed in one of the preceding claims,
characterized in that
R¹ contains an uneven number of C atoms.

14. The drug formulation as claimed in one of claims 1 to 7,
characterized in that
R¹ is a hexadecyl, octadecyl, oleyl, elaidyl, eicosyl, eicosenyl-cis-(ω-9), heneicosyl, heneicosenyl, docosyl or docosenyl radical.

15. The drug formulation as claimed in one of the preceding claims,
characterized in that
R¹ is a doubly unsaturated hydrocarbon radical containing cis double bonds in an unconjugated position.

16. The drug formulation as claimed in one of the preceding claims,
characterized in that
R², R³ and R⁴ are methyl radicals.

17. The drug formulation as claimed in one of the preceding claims,
characterized in that
it is present in a form which is suitable for intravenous administration.

18. The drug formulation as claimed in one of claims 1 to 16,
characterized in that
it is present in a form which is suitable for oral administration.

19. A process for producing a drug formulation as claimed in one of claims 1 to 18,
characterized in that
a) from 30 to 60 mol % of a phospholipid compound of the formula I: 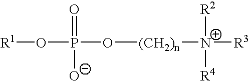
in which
R¹ is a saturated or unsaturated hydrocarbon radical having from 16 to 24 C atoms,
R², R³ and R⁴ are, in each case independently, H, a C₁-C₅-alkyl group, a C₃-C₆-cycloalkyl group or a C₁-C₅-hydroxyalkyl group, with two of R², R³ and R⁴ being able to form, with each other, a C₂-C₅-alkylene group which can optionally be substituted by an —O—, —S— or NR⁵ group, in which R⁵ is H, a C₁-C₅-alkyl group, a C₃-C₆-cycloalkyl group or a C₁-C₅-hydroxyalkyl group,
and n is an integer from 2 to 4,
are mixed in aqueous solution, with b) from 25 to 65 mol % of cholesterol and/or a cholesterol derivative and c) from 5 to 15 mol % of a phosphatidylmonoglycerol or phosphatidyloligoglycerol, with a), b) and c) together comprising 100 mol %, and a water-miscible, physiologically acceptable alcohol having from 2 to 4 C atoms is added to the resulting mixture, such that the components form a complex which is dispersed in water.

20. The process as claimed in claim 19,
characterized in that
the alcohol is added while heating to from 20° C. to 85° C.

21. The process as claimed in claim 19 or 20,
characterized in that
phosphatidylmonoglycerols or phosphatidyloligoglycerols containing from 1 to 4 glycerol derivatives are used as component c).
